(19) United States
(12) Patent Application Publication
(10) Pub. No.: US 2025/0257244 A1
(43) Pub. Date: Aug. 14, 2025

(54) REINFORCED ADHESIVE FILM, IN PARTICULAR FOR APPLICATIONS IN THE MEDICAL FIELD, AND METHOD FOR MANUFACTURING SUCH A FILM

(71) Applicant: ADHEX TECHNOLOGIES, Chenôve (FR)

(72) Inventors: Maud WILLIAMS, DIJON (FR); Julie MATRAY, FAUVERNEY (FR)

(21) Appl. No.: 19/048,531

(22) Filed: Feb. 7, 2025

(30) Foreign Application Priority Data
Feb. 9, 2024 (FR) 2401287

(51) Int. Cl. C09J 7/21; A61B 46/00; A61B 46/20; C09J 109/06; C09J 133/08

(52) U.S. Cl. CPC C09J 7/21; A61B 46/40; C09J 109/06; C09J 133/08; A61B 2046/205; C09J 2203/358; C09J 2301/124; C09J 2301/302; C09J 2400/26

(57) ABSTRACT

A reinforced adhesive film, in particular for applications in the medical field, and method for manufacturing such a film is disclosed. The method for manufacturing a reinforced adhesive film comprises a single main coating step, implemented by a coating device, consisting, in a single pass, in completely embedding a weft formed from a non-woven fabric in a pressure-sensitive adhesive formulation so that the adhesive formulation completely covers both faces of the weft, which allows to obtain rapidly, using a simple manufacturing method, a reinforced adhesive film with advantageous properties in particular in terms of adhesion, strength, conformability and breathability.

### CROSS-REFERENCE TO RELATED APPLICATION

This present application claims priority to French Patent Application No. 2401287, filed Feb. 9, 2024, the disclosures of which are hereby incorporated by reference in their entireties.

### TECHNICAL FIELD

The present disclosure relates to a reinforced adhesive film, in particular for applications in the medical field, and to a method for manufacturing such a film.

### BACKGROUND

The reinforced adhesive film may be used in various fields, in particular in the industrial field and especially in the building sector, as well as in the medical field, especially for the adhesion of any type of operating field.

This adhesive film, when used in a hospital operating theatre, may be applied by surgeons during operations, for bonding either to an operating table or directly to a patient.

There are currently double-sided adhesives, incorporating adhesive formulations, which are used for such applications.

On their own, such adhesive formulations have a number of disadvantages. In particular:
it is necessary to make them more consistent, solid and resistant, particularly for use in the medical field, which tends to reduce and limit the extent of their properties; and
when they are cut for use, they tend to pollute the cutting tools.

The double-sided adhesives, on the other hand, are resistant during the cutting and use methods, but have the disadvantage of being non-permeable to water, which may lead to delamination when applied to different substrates.

In addition, the presence of a central weft makes the assembly rigid and reduces the adhesive performance of the laminate.

There is therefore an interest and a need for a reinforced adhesive film allowing to overcome these disadvantages.

### SUMMARY

The purpose of this disclosure is to provide a reinforced adhesive film that may be used in particular in the above-mentioned applications and to meet this need.

According to the disclosure, said reinforced adhesive film comprises a weft formed from a non-woven fabric, which is completely embedded in a pressure-sensitive adhesive formulation such that the adhesive formulation (completely encompasses the weft and) completely covers both faces of the weft.

The result is an adhesive film with enhanced strength and resistance, thanks to the integration of the weft (formed from the non-woven fabric). This also ensures that the initial properties and performance of the adhesive film reinforced in this way do not deteriorate over time, particularly when it is subjected to various processing steps as is generally the case.

The addition of the weft to the adhesive formulation not only reinforce the strength of the adhesive film, but also improves its creep stability during storage of the finished product comprising this adhesive film and makes it easier to cut the film (without polluting the cutting tools).

The non-occlusive nature of said adhesive film is also an advantage when it comes to moisture strength on different surfaces.

Thanks to its advantageous properties described below, this reinforced adhesive film is particularly well suited to use in the medical field, especially in an operating theatre for application by surgeons during operations, by bonding either to an operating table or directly to a patient. This reinforced adhesive film may be used to advantage as a replacement for conventional double-sided adhesives, eliminating the disadvantages mentioned above.

In a preferred embodiment, the reinforced adhesive film has at least some of the following characteristics:
a moisture vapor transmission rate, measured according to the measurement method of standard NF EN 13726-2, greater than 400 g/m²/24 h, illustrating its breathability properties;
an adhesive strength at 90° for 1 minute on glass greater than 300 N/m; and
an increased conformability (compared in particular with double-sided adhesives), as described below.

In addition, the non-woven fabric forming the weft has a grammage of between 3 and 8 g/m², measured using the measurement method of standard FTM12.

These characteristics allow the film to achieve the above properties.

In a first embodiment, the adhesive formulation is a cross-linkable acrylic polymer under ultraviolet light with butyl acrylate and acrylic acid as the base monomers.

Preferably, this acrylic polymer is formulated with a tackifying resin of hydrocarbon nature.

In a second embodiment, the adhesive formulation is an acrylic polymer in solvent-phase with 2-ethylhexylacrylate as the base monomer.

In a third embodiment, the adhesive formulation is a styrene-isoprene-styrene or styrene-butadiene-styrene elastomer.

Preferably, the elastomer is formulated with at least one of the following types of elements: resin, plasticizer, antioxidant.

Advantageously, the non-woven fabric forming the weft is made of polyester and has at least some of the following characteristics:
a thickness of between 0.03 and 0.07 mm, measured using the measurement method of standard NF EN ISO 534;
a resistance at break of between 20 and 30 N/5 cm, measured using the measurement method of standard NF EN 29073-3;
an elongation at break of between 4 and 8%, measured according to the measurement method in standard NF EN 29073-3; and
a moisture vapor transmission rate greater than 10,000 g/m²/24 h, measured using the measurement method of standard NF EN 13726-2.

Preferably, the fibers of the non-woven fabric forming the weft are oriented substantially in one and the same direction so as to increase the resistance of the adhesive film.

In addition, in a preferred embodiment, the reinforced adhesive film comprises a protective film which is silicone-coated on its two faces and which supports on one of its silicone-coated faces the assembly formed by the adhesive formulation wherein the weft is embedded.

The disclosure also relates to a method for manufacturing a reinforced adhesive film.

According to the disclosure, said method comprises a single main coating step, implemented by a coating device, consisting, in a single pass, in completely embedding a weft formed from a non-woven fabric in a pressure-sensitive adhesive formulation so that the adhesive formulation (completely encompasses the weft and) completely covers both faces of the weft. In addition, said reinforced adhesive film has a moisture vapor transmission rate, measured according to standard NF EN 13726-2, greater than 400 g/m²/24 h, an adhesive strength at 90° for 1 minute on glass greater than 300 N/m, and increased conformability compared with double-sided adhesives, and the non-woven fabric forming the weft has a grammage, measured according to FTM12, of between 3 and 8 g/m².

Thus, thanks to the disclosure, the reinforced adhesive film (which has the above-mentioned advantageous characteristics) is obtained in a single pass during which the non-woven fabric is completely embedded in the adhesive formulation. This allows to obtain a particularly simple manufacturing method and rapid manufacturing, compared with conventional methods which comprise at least two passes (i.e. a first pass for depositing an adhesive on a first face of a support and a second pass for depositing an adhesive on the second face of the support).

Advantageously, the non-woven fabric of the weft has suitable characteristics to allow the adhesive formulation to pass through it and to be present on both sides of the non-woven fabric. These characteristics are preferably adapted to the characteristics of the adhesive formulation.

The method also comprises an auxiliary coating step which consists in depositing the adhesive formulation on a protective film, and which:
in a first embodiment, is implemented prior to the main coating step; and
in a second embodiment, is implemented at the same time as the main coating step.

Advantageously, the adhesive formulation used is either hotmelt or solvent-phase.

### BRIEF DESCRIPTION OF THE FIGURES

Other advantages and characteristics will be clearer from the following description of several non-limiting embodiments of a reinforced adhesive film conforming to the disclosure and its manufacturing method, with particular reference to the appended figures. In these figures, identical references designate similar elements.

FIG. 1 is a partial schematic cross-sectional view of a reinforced adhesive film in accordance with one embodiment of the disclosure.

FIG. 2 is a schematic view of a first embodiment of a device for manufacturing a reinforced adhesive film.

FIG. 3 is a schematic view of a second embodiment of a device for manufacturing a reinforced adhesive film.

FIG. 4 is a schematic view of a variant of the second embodiment shown in FIG. 3.

### DETAILED DESCRIPTION

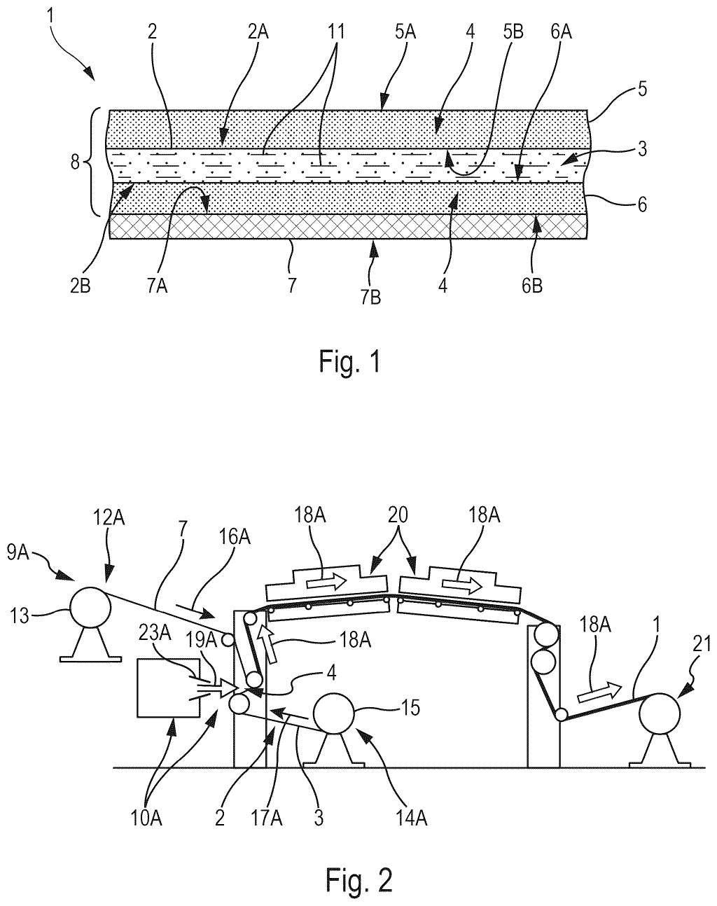

The film 1 used to illustrate the disclosure and shown in part according to a particular embodiment in FIG. 1 is a reinforced adhesive film, as described below.

As shown in FIG. 1, the film 1 comprises a weft 2 formed from a non-woven fabric 3.

This weft 2 is completely embedded in a pressure-sensitive adhesive formulation 4 so that the adhesive formulation 4 completely encompasses the weft 2 and completely covers both faces 2A and 2B of the weft 2. Two adhesive layers 5 and 6 are formed on either side of the weft 2 from non-woven fabric 3. The adhesive layer 5 is formed on the face 2A of the weft 2 and the adhesive layer 6 is formed on the face 2B of the weft 2.

As explained below, the adhesive formulation 4 used to manufacture the film 1 is either hotmelt or solvent-phase. The adhesive formulation 4 is represented schematically by small black dots in FIGS. 1, 3 and 4.

The film 1 also comprises a protective film 7 on one face of the assembly 8 formed by the adhesive formulation 4 wherein the weft 2 is embedded.

In the example shown in FIG. 1, the protective film 7 is in contact with one face 7A of a face 6B of the adhesive layer 6 (which is opposite the face 6A in contact with the face 2B of the weft 2). The adhesive layer 5 comprises two faces 5A and 5B, of which the face 5B is in contact with the face 2A of the weft 2.

Preferably, the protective film 7 is a glassine paper, siliconized on both faces 7A and 7B. This allows the film 1 to be wound on itself, with the face 5A of the film 1 coming into contact with the (siliconized) face 7B of the protective film 7 during winding.

In a preferred embodiment, the reinforced adhesive film 1 has at least some of the following characteristics:
an adhesive strength at 90° for 1 minute on glass, greater than 300 N/m on the visible face and hidden face, illustrating its adhesion properties;
a T-shaped decomplexing force between the protective film 7 and the adhesive layer 6 of between 10 and 60 cN/5 cm;
a moisture vapor transmission rate (MVTR), i.e. the rate at which water permeates through a membrane, measured (according to the measurement method of standard NF EN 13726-2) in mass per unit area per unit time, in this case in g/m²/24 h, which is greater than 400 g/m²/24 h (hidden face and visible face), illustrating its breathability properties;
a T-shaped decomplexing force on the operating field (film) after 24 hours, greater than 500 cN/5 cm;
a T-shaped decomplexing force on the operating field (non-woven fabric) after 24 hours, greater than 500 cN/5 cm.

In the context of this disclosure, the ranges of values presented in the form of “between x and y” include the limits x and y, the integers between these limits, as well as all the other real numbers between these limits.

The result is an adhesive film 1 whose strength and resistance are enhanced by the integration of the weft 2 (formed from the non-woven fabric 3). In addition, the initial properties and performance of the adhesive film 1 reinforced in this way do not deteriorate over time, particularly when it is subjected to various processing steps as is generally the case. Adding the weft 2 to the adhesive formulation 4 also allows to improve its creep stability during storage of the finished product comprising this film 1, and makes it easier to cut the film 1 (without polluting the cutting tools used for this purpose).

In the context of the present disclosure, the adhesive formulation 4 used to manufacture the film 1 may be produced in different ways.

In a first embodiment, the adhesive formulation 4 corresponds to an acrylic polymer cross-linkable under ultraviolet (UV) light, with butyl acrylate (BA) and acrylic acid (AA) as the base monomers.

Preferably, this acrylic polymer is formulated with a tackifying resin of hydrocarbon nature, in order to provide a particular adhesiveness to the film 1 (if necessary or if useful).

In a second embodiment, the adhesive formulation 4 is a solvent-phase acrylic polymer, non-cross-linkable under ultraviolet light, based on the monomer EHA (2-ethylhexyl acrylate).

In a third embodiment, the adhesive formulation 4 is a styrene-isoprene-styrene (SIS) or styrene-butadiene-styrene (SBS) elastomer.

Preferably, this elastomer is formulated with at least one of the following types of elements: resin, plasticizer, antioxidant, in order to provide the desired properties to the film 1.

It should be noted that the choice of the chemical nature (acrylic or elastomer) of the adhesive formulation 4 may depend on the substrate or substrates of the intended application and the application time of the adhesive formulation 4.

By way of illustration, the table below shows the main characteristics of the adhesive formulation 4 for four different examples, named FA1 to FA4, which may be used to make the film 1.

| | | | | |
|---|---|---|---|---|
| Commercial | FA1: HM-R2292 | FA2: HM-R2519A | FA3: AS-333 | FA4: AS-1959 |
| reference | | | | |
| Technology | hotmelt | hotmelt | solvent-phase | solvent-phase |
| Composition | 94% butyl | 89.3% butyl | 2-ethylhexyl | 2-ethylhexyl |
| of monomers | acrylate | acrylate | acrylate | acrylate |
| making up | 4% acrylic acid | 3.8% acrylic acid | vinyl acetate | vinyl acetate |
| the acrylic | 2% ethyl acrylate | 1.9% ethyl acrylate | acrylic acid | acrylic acid |
| (%) | | 5% aromatic | | |
| | | tackifier resin | | |
| Brookfield | 130° C.: | 130° C.: | 25° C.: | 25° C.: |
| viscosity | speed 1: 75,000 | speed 1: 54,750 | 3,200-5,800 | 4,000-10,000 |
| (mPa · s) | speed 2.5: | speed 2.5: 47,100 | | |
| | 71,000 | speed 5: 43,550 | | |
| | speed 5: 66,000 | | | |

This table shows the adhesive formulation 4 considered for the four examples FA1 to FA4:
their commercial reference;
the technology (hotmelt or solvent-phase);
the composition of the monomers making up the acrylic, in percent (%); and
the Brookfield viscosity in mPa·s (measured using the measurement method of standard NF EN ISO 2555).

In a preferred embodiment, the non-woven fabric 3 forming the weft 2 is very aerated, in particular to allow the adhesive formulation 4 to pass through. Preferably, the non-woven fabric 3 is made of polyester fibers.

In a preferred embodiment, the fibers 11 of the non-woven fabric 3 are of the monodirectional type and are therefore oriented in one and the same direction. In particular, this allows to increase the resistance of the film 1.

Compared with spunbond technology non-woven fabric, thinner thicknesses may be achieved for the non-woven fabric 3 with the same grammage.

In a preferred embodiment, the non-woven fabric 3 has at least some of the following characteristics:
a grammage of between 3 and 8 g/m² (measured using the measurement method of standard FTM12 (FINAT Test Method);
a thickness of between 0.03 and 0.07 mm (measured using the measurement method of standard NF EN ISO 534);
a resistance at break of between 20 and 30 N/5 cm (measured using the measurement method of standard NF EN 29073-3) in the machine direction (MD for machine direction);
an elongation at break of between 4 and 8% (measured using the measurement method of standard NF EN 29073-3);
a Moisture Vapor Transmission Rate (MVTR), i.e. the rate at which water permeates through a membrane, measured (using the measurement method of standard NF EN 13726-2) in mass per unit area per unit time, in this case in g/m²/24 h, which is greater than 10,000 g/m²/24 h.

The film 1, as described above, may be obtained using a manufacturing method specified below. This manufacturing method, preferably for large widths, includes a main coating step in a single pass.

This (single) main coating step, which is implemented by a coating device, consists, in a single pass, in completely embedding a weft 2 formed from a non-woven fabric 3 in a pressure-sensitive adhesive formulation 4 so that the adhesive formulation 4 completely encompasses the weft 2 and completely covers both faces 2A and 2B of the weft 2 (FIG. 1).

In this way, the (reinforced adhesive) film 1 with the above-mentioned advantageous characteristics is obtained in a single pass (at the level of the coating device), after which the non-woven fabric 3 is completely embedded in the adhesive formulation 4.

To do this, the non-woven fabric 3 has suitable characteristics and is in particular highly aerated to allow the adhesive formulation 4 to pass through it and be present on both sides of the non-woven fabric 3, for example when the non-woven fabric 3 passes under a coating nozzle (feeding the adhesive formulation 4) of the coating device. These characteristics of the non-woven fabric 3 of the weft 2 (such as being aerated) may be adapted to the characteristics (e.g. viscosity) of the adhesive formulation 4 to allow the adhesive formulation 4 to be present on both sides of the weft 2 and to completely encompass it.

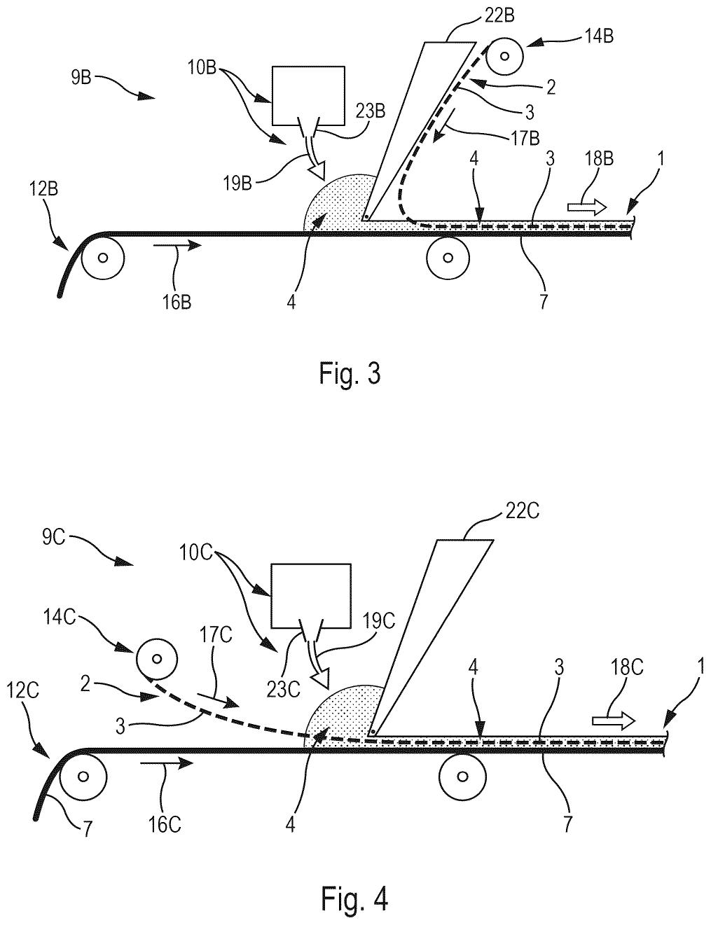

The manufacturing method also comprises an “auxiliary” coating step, consisting in depositing the adhesive formulation 4 on the protective film 7. The weft 2 may be embedded in the adhesive formulation 4 at the same time as the latter is deposited on the protective film 7 or after the adhesive formulation 4 has been deposited on the protective film 7. Thus, in a first implementation corresponding to the embodiments of FIGS. 2 and 4 described below, the auxiliary coating step is implemented at the same time as the main coating step, and in a second implementation corresponding to the embodiment of FIG. 3, the auxiliary coating step (deposition of the adhesive formulation 4 on the protective film 7) is implemented prior to the main coating step (integration of the weft 2 into the adhesive formulation 4).

In the context of the present disclosure, the manufacturing method, and in particular its main coating step, may be implemented using various coating devices such as, for example, those described below, by way of illustration, with reference to FIGS. 2 to 4.

In a first embodiment shown in FIG. 2, the manufacturing method uses a manufacturing device 9A which comprises a coating device 10A. The manufacturing device 9A is configured to use a “hotmelt” method in a large width (1500 mm).

In this first embodiment, the manufacturing device 9A also comprises a supply system 12A supplying protective film 7, provided in particular with a reel 13 of protective film 7, and a supply system 14A for supplying non-woven fabric 3, provided in particular with a reel 15 of non-woven fabric 3.

The non-woven fabric 3 and the protective film 7 are fed by these supply systems 12A and 14A, in the directions illustrated by arrows 16A and 17A respectively, to the coating device 10A. The coating device 10A is provided with at least one coating nozzle 23A and is supplied, in the usual way, with adhesive formulation 4. The protective film 7, which serves as a support for the hotmelt adhesive formulation 4 wherein the weft 2 from non-woven fabric 3 is embedded (also fed at this point), passes under the coating nozzle or nozzles 16A (to receive the adhesive formulation 4 as illustrated by an arrow 19A) so that, in relation to the characteristics of the non-woven fabric 3, the hotmelt adhesive formulation 4 is distributed on both sides of the weft 2 from non-woven fabric 3.

The assembly (formed by this non-woven fabric 3 which is embedded in the adhesive formulation 4 deposited to the protective film 7) is fed, in the direction illustrated by arrows 18A, through a drying system 20 (or drying tunnel or oven or ovens), then wound at a winding station 21, downstream of the drying system 20. At this winding station 21, the reinforced adhesive film 1 just manufactured is wound onto a reel.

In an alternative embodiment (not shown), the drying system 20 is replaced in the usual way by UV lamps, particularly when hotmelt acrylics which may be cross-linked under ultraviolet (UV) light are used for the adhesive formulation 4.

In a second embodiment shown in FIG. 3, the manufacturing method uses a manufacturing device 9B comprising a coating device 10B configured to implement a solvent-phase manufacturing, in a large width (1500 mm).

In this second embodiment, the manufacturing device 9B also comprises a supply system 12B for supplying protective film 7 and a supply system 14B for supplying non-woven fabric 3.

The non-woven fabric 3 and the protective film 7 are fed by these supply systems 12B and 14B, in the directions illustrated by arrows 16B and 17B respectively, to the coating device 10B. The coating device 10B is provided with at least one supply system 23B which supplies the adhesive formulation 4 in the usual way.

The coating device 10B is configured to deposit the adhesive formulation 4 in solvent-phase on the protective film 7, as illustrated by an arrow 19B, upstream of a squeegee 22B of the coating device 10B, in the direction of movement (illustrated by an arrow 18B) of the film 1.

The non-woven fabric 3 (supplied by the non-woven fabric supply system 14B) is integrated downstream of the squeegee 22B, in the adhesive formulation 4 previously deposited on the protective film 7.

Downstream of this integration, the assembly (formed by this non-woven fabric 3 which is embedded in the adhesive formulation 4 deposited on the protective film 7) is subjected to conventional treatments, in particular drying and winding, using conventional devices (not shown).

Furthermore, in a third embodiment shown in FIG. 4, which is a variant of the second embodiment shown in FIG. 3, the manufacturing method uses a manufacturing device 9C comprising a coating device 10C. Unlike the coating device 10B of the second embodiment, the coating device 10C is configured so that the non-woven fabric 3 is integrated into the adhesive formulation 4 deposited on the protective film 7, upstream of a squeegee 22C in the direction of movement (illustrated by an arrow 18C) of the film 1.

The supply system 14C is therefore arranged upstream of the squeegee 22C. Most of the other elements of the manufacturing device 9C are identical or similar to those of the manufacturing device 9B. In particular, the non-woven fabric 3 and the protective film 7 are fed by supply systems 12C and 14C, in the direction illustrated by arrows 16C and 17C respectively, to the coating device 10C provided with at least one supply system 23C supplying the adhesive formulation 4 (arrow 19C).

Whichever coating device 10A, 10B and 10C and manufacturing device 9A, 9B and 9C are used, the implemented manufacturing method uses a non-woven fabric 3, an adhesive formulation 4 and a protective film 7, such as those described above.

In particular, it takes into account the above-mentioned characteristics (such as thickness, grammage, etc.) of the non-woven fabric 3, the above-mentioned characteristics of the adhesive formulation 4 and the above-mentioned characteristics of the protective film 7.

The manufacturing devices 9A, 9B and 9C described above allow a large-width coating of an adhesive formulation 4 on a non-woven fabric 3 in a single pass. This provides a simple and rapid manufacturing method, compared with conventional manufacturing methods which involve at least two passes (i.e. a first pass for depositing an adhesive on a first face of a substrate and a second pass for depositing an adhesive on the second face of the substrate).

In addition, the reinforced adhesive film 1 obtained in this way has the particularly advantageous characteristics described above.

The table below shows a comparison between an example Fl of the film 1 (obtained by a method in accordance with a particular embodiment of the disclosure) and a known conventional double-sided adhesive (P7592) and allows to highlight the advantageous characteristics of the film 1.

| | F1 | | P7592 |
|---|---|---|---|
| Construction | Adhesive | HM-R 2519A, 60 g/m² | visible face: HMR-2292, 60 g/m² |
| | | | hidden face: HMR-2292, 40 g/m² |
| | Weft | monodirectional non-woven | none |
| | | fabric | |
| | Support | none | film PET, 17 g/m² |
| | Protective | differentiated siliconized | non-differentiated siliconized paper |
| | film | paper 2F | 2F |

| | | |
|---|---|---|
| Grammage | 60 | 100 |
| g/m² | | |
| AS at 90° for 1 minute on | 467 | 750 |
| glass - Visible face - cN/cm | | |
| AS at 90° for 1 minute on | 565 | 825 |
| glass - Hidden face - cN/cm | | |
| MVTR vapor contact | 870 | 45 |
| (g/m²/24 h) | | |

This table shows the two products under consideration:
the characteristics of the adhesive (or adhesive formulation) used;
the presence or absence of a weft and of a support;
the characteristics of the protective film used;
the grammage (measured using the measurement method of standard FTM12) in g/m²;
the adhesive strength AS at 90° for 1 minute on glass, for the visible face and the hidden face respectively, in cN/cm; and
the moisture vapor transmission rate (MVTR), measured in accordance with standard NF EN 13726-2, in g/m²/24 h.

This table allows to highlight the following advantageous characteristics of the film 1:
a moisture vapor transmission rate greater than 400 g/m²/24 h, illustrating these breathability properties;
an adhesive strength greater than 300 N/m; and
superior conformability compared to double-sided adhesives.

The improved conformability of the film 1 (i.e. superior to that of double-sided adhesives) may be demonstrated by a mechanical hysteresis test and a test illustrating flexibility.

With regard to the hysteresis test, deformation measurements at 20% were carried out both on the film 1 and on the usual double-sided adhesive mentioned above (P7592), for test specimens 15 mm wide and 10 cm long.

The table below shows a comparison between the example F1 of film 1 (obtained by a method in accordance with a particular embodiment of the disclosure) and the usual known double-sided adhesive (P7592), showing the values of various parameters, and allows to highlight the advantageous characteristics of the film 1 with regard to the conformability.

| | Hysteresis force | Remanence | Slope at start |
|---|---|---|---|
| | (N/cm) | (%) | (N/cm/%) |
| F1 | 6.4 | 14.9 | 0.6 |
| P7592 | 13.5 | 15.4 | 4.5 |

This table shows for the two products under consideration:
the hysteresis force, in N/cm;
the remanence, in %; and
the slope at the start of the hysteresis curve, in N/cm/%.

The difference between the measurements in terms of remanence is small, but the double-sided adhesive is more resistant to the deformation than the film 1.

As for the slopes at the start of the hysteresis curves (and therefore the Young's moduli), they show a difference in behavior between the two products analyzed. The double-sided adhesive is much stiffer than the film 1.

In addition, the test specimens 20 cm long and 2 cm wide were used for the loop flexibility test. For each of the two products, the corresponding test tube was held by its free ends, brought into contact and arranged vertically upwards, so as to form a loop.

In this test, we observe a different loop shape for the two products, with a maximum loop width:
2.5 cm for the film 1; and
4 cm for the standard double-sided adhesive (P7592).

This test allows to demonstrate the flexibility of the film 1 (reinforced by the weft 2) compared with the double-sided adhesive (polyester).

The improved conformability of the film 1, compared with a conventional double-sided adhesive, was demonstrated by these two tests, the mechanical hysteresis test and the flexibility test.

The film 1, as described above, which has advantageous adhesive and cohesive properties, is particularly well suited to use in an operating theatre, and may in particular be applied by surgeons during operations in a hospital environment, for bonding either to an operating table or directly to a patient.

The film 1, as described above, may be used in many other applications, particularly (but not exclusively) in the medical sector. More generally, the film 1 may be used in all applications where its advantageous characteristics (and in particular its reinforced adhesive properties) are required. In particular, it may be used in the industrial applications, especially in the construction sector, where its breathability properties may be exploited to the full.

It is clear that the examples presented above are only specific illustrations, and in no way limit the fields of application of this disclosure. In addition, characteristics of some of these different examples may be combined with each other where appropriate, without departing from the scope of this disclosure.

1. A reinforced adhesive film for a medical field, comprising a weft formed from a non-woven fabric, which is completely embedded in a pressure-sensitive adhesive formulation such that the adhesive formulation completely covers both faces of the weft, said reinforced adhesive film having a moisture vapor transmission rate, measured according to standard NF EN 13726-2, greater than 400 g/m²/24 h, an adhesive strength at 90° for 1 minute on glass greater than 300 N/m, and an increased conformability compared with double-sided adhesives, the non-woven fabric forming the weft having a grammage, measured according to FTM12, of between 3 and 8 g/m².

2. The film according to claim 1, wherein the adhesive formulation is a cross-linkable acrylic polymer under ultraviolet light with butyl acrylate and acrylic acid as base monomers.

3. The film according to claim 2, wherein the acrylic polymer is formulated with a tackifying resin of hydrocarbon nature.

4. The film according to claim 1, wherein the adhesive formulation is an acrylic polymer in solvent-phase, with 2-ethylhexylacrylate as a base monomer.

5. The film according to claim 1, wherein the adhesive formulation is a styrene-isoprene-styrene or styrene-butadiene-styrene elastomer.

6. The film according to claim 5, wherein the elastomer is formulated with at least one of the following types of element: resin, plasticizer, antioxidant.

7. The film according to claim 1, wherein the non-woven fabric forming the weft is made of polyester, and has at least some of the following characteristics:
a thickness of between 0.03 and 0.07 mm, measured in accordance with standard NF EN ISO 534;
a resistance at break of between 20 and 30 N/5 cm, measured in accordance with a standard NF EN 29073-3;
an elongation at break of between 4 and 8%, measured in accordance with the standard NF EN 29073-3; and
a moisture vapor transmission rate of over 10,000 g/m²/24 h, measured in accordance with the standard NF EN 13726-2.

8. The film according to claim 1, wherein fibers of the non-woven forming the weft are oriented substantially in one and the same direction.

9. The film according to claim 1, wherein it comprises a protective film which is silicone-coated on its two faces and which supports, on one of its silicone-coated faces, the assembly formed by the adhesive formulation wherein the weft is embedded.

10. A method for manufacturing a reinforced adhesive film, wherein it comprises a single main coating step, implemented by a coating device, consisting, in a single pass, in completely embedding a weft formed from a non-woven fabric in a pressure-sensitive adhesive formulation so that the adhesive formulation completely covers both faces of the weft, said reinforced adhesive film having a moisture vapor transmission rate, measured according to standard NF EN 13726-2, greater than 400 g/m²/24 h, an adhesive strength at 90° for 1 minute on glass greater than 300 N/m, and an increased conformability compared with double-sided adhesives, the non-woven fabric forming the weft having a grammage, measured according to FTM12, of between 3 and 8 g/m².

11. The method according to claim 10, wherein it comprises an auxiliary coating step consisting in depositing the adhesive formulation on a protective film, which is implemented prior to the main coating step.

12. The method according to claim 10, wherein it comprises an auxiliary coating step consisting in depositing the adhesive formulation on a protective film, which is implemented at the same time as the main coating step.

13. The method according to claim 10, wherein the adhesive formulation used is either hotmelt or solvent-phase.
